(19) United States
(12) Patent Application Publication
(10) Pub. No.: US 2024/0090879 A1
(43) Pub. Date: Mar. 21, 2024

(54) INFORMATION PROCESSING METHOD, INFORMATION PROCESSING DEVICE, AND NON-TRANSITORY COMPUTER READABLE RECORDING MEDIUM STORING INFORMATION PROCESSING PROGRAM

(71) Applicant: PANASONIC HOLDINGS CORPORATION, Osaka (JP)

(72) Inventors: Hirofumi KANAI, Osaka (JP); Yuka YAMADA, Nara (JP); Hideyuki MAEHARA, Osaka (JP)

(73) Assignee: PANASONIC HOLDINGS CORPORATION, Osaka (JP)

(21) Appl. No.: 18/525,223

(22) Filed: Nov. 30, 2023

Related U.S. Application Data
(63) Continuation of application No. PCT/JP2021/038439, filed on Oct. 18, 2021.
(60) Provisional application No. 63/195,403, filed on Jun. 1, 2021.

(30) Foreign Application Priority Data
Oct. 6, 2021 (JP) 2021-164550

(51) Int. Cl. A61B 10/00; A61B 5/00

(52) U.S. Cl. CPC A61B 10/0038; A61B 5/746; A61B 10/007

(57) ABSTRACT

A server acquires, in a first predetermined period, first data in which excretion related data of a user, acquired by an excretion sensor installed in a toilet, and a user ID for identifying the user are associated with each other, generates a reference database indicating an excretion tendency of the user on the basis of the acquired first data, acquires, in a second predetermined period, second data in which excretion related data of the user, acquired by the excretion sensor installed in the toilet, and the user ID are associated with each other, generates alert information for the user on the basis of the reference database and the second data, and outputs the generated alert information.

### TECHNICAL FIELD

The present disclosure relates to a technique for outputting alert information to the user.

### BACKGROUND ART

For example, Patent Literature 1 discloses that a biological data acquisition device identifies the user, acquires biological data of the user from excrement excreted from the user in a toilet, acquires a transmission destination associated with the identified user from a storage part that stores any analysis processing device as a transmission destination in association with the user, and transmits the biological data to the acquired transmission destination. Further, Patent Literature 1 discloses that an analysis processing device transmits a main analysis result obtained by analyzing biological data acquired from the biological data acquisition device to the biological data acquisition device, and the biological data acquisition device receives the main analysis result transmitted from the analysis processing device and presents the main analysis result to the user.

However, in the above-described conventional technique, a past excretion tendency of the user is not considered, and it is difficult to output alert information according to a change in a health state of the individual user, and further improvement has been required.

### CITATION LIST

### Patent Literature

Patent Literature 1: JP 2019-150139 A

### SUMMARY OF INVENTION

The present disclosure has been made to solve the above problem, and an object of the present disclosure is to provide a technique capable of outputting alert information according to a change in a health condition of the individual user.

An information processing method according to the present disclosure includes, by a computer, acquiring first data in which excretion related data of a user acquired by a sensor installed in a toilet and a user ID for identifying the user are associated with each other in a first predetermined period, generating a reference database indicating an excretion tendency of the user based on the acquired first data, acquiring second data in which the excretion related data of the user acquired by the sensor installed in the toilet and the user ID are associated with each other in a second predetermined period, generating alert information to the user based on the reference database and the second data, and outputting the generated alert information.

According to the present disclosure, it is possible to detect a change in an excretion tendency of the user himself or herself and output alert information according to a change in a health condition of an individual user.

### BRIEF DESCRIPTION OF DRAWINGS

FIG. 1 is a diagram illustrating a configuration of an excretion management system in an embodiment of the present disclosure.

FIG. 2 is a diagram for explaining arrangement positions of an excretion sensor and an excrement determination device in the embodiment of the present disclosure.

FIG. 3 is a block diagram illustrating an example of a configuration of a server according to the embodiment of the present disclosure.

FIG. 4 is a first flowchart illustrating an example of processing of the server according to the embodiment of the present disclosure.

FIG. 5 is a second flowchart illustrating an example of processing of the server according to the embodiment of the present disclosure.

FIG. 6 is a third flowchart illustrating an example of processing of the server according to the embodiment of the present disclosure.

FIG. 7 is a diagram illustrating an example of a display screen of fourth alert information displayed on a display device.

FIG. 8 is a diagram illustrating an example of a display screen of seventh alert information displayed on the display device.

FIG. 9 is a diagram illustrating an example of a display screen of eighth alert information displayed on the display device.

### DESCRIPTION OF EMBODIMENTS

(Knowledge Underlying Present Disclosure)

In the above-described conventional technique, abnormality detection is performed by comparison with a predetermined normal range based on statistical data, and output of alert information in consideration of daily excretion rhythm of the user is not disclosed. Further, details of a timing of outputting alert information for each risk are not disclosed, and alert information is possibly not output at an appropriate timing depending on a risk.

In order to solve the above problem, an information processing method according to an aspect of the present disclosure includes, by a computer, acquiring first data in which excretion related data of a user acquired by a sensor installed in a toilet and a user ID for identifying the user are associated with each other in a first predetermined period, generating a reference database indicating an excretion tendency of the user based on the acquired first data, acquiring second data in which the excretion related data of the user acquired by the sensor installed in the toilet and the user ID are associated with each other in a second predetermined period, generating alert information to the user based on the reference database and the second data, and outputting the generated alert information.

According to this configuration, first, in the first predetermined period, first data in which excretion related data of the user acquired by the sensor installed in a toilet is associated with a user ID for identifying the user is acquired. Then, a reference database indicating an excretion tendency of the user is generated on the basis of the first data acquired in the first predetermined period. After the above, in the second predetermined period, second data in which excretion related data of the user acquired by the sensor installed in a toilet is associated with a user ID is acquired. Then, alert information to the user is generated on the basis of the reference database and the second data acquired in the second predetermined period, and the generated alert information is output.

Therefore, general statistical data and a current excretion tendency of the user in the second predetermined period are not compared, and a past excretion tendency of the user in the first predetermined period and a current excretion tendency of the user in the second predetermined period are compared. Accordingly, it is possible to detect a change in an excretion tendency of the user himself or herself and output alert information according to a change in a health condition of an individual user.

Further, in the above information processing method, the excretion related data may include feces shape data indicating whether a shape of excreted feces is hard feces, normal feces, or watery feces, the reference database may include a first hard feces ratio indicating a ratio of the hard feces excreted among all evacuations in the first predetermined period, and in generation of the alert information, a second hard feces ratio indicating a ratio of the hard feces excreted among all evacuations in the second predetermined period may be calculated, and in a case where the second hard feces ratio is higher than the first hard feces ratio, first alert information indicating that the user is highly likely to have constipation may be generated.

According to this configuration, the first hard feces ratio indicating a ratio of hard feces excreted among all evacuations in the first predetermined period is compared with the second hard feces ratio indicating a ratio of hard feces excreted among all evacuations in the second predetermined period. Therefore, the first alert information indicating that the user is highly likely to have constipation can be output according to a change in a shape of feces of an individual user.

Further, in the above information processing method, the excretion related data may include feces shape data indicating whether a shape of excreted feces is hard feces, normal feces, or watery feces, the reference database may include a first watery feces ratio indicating a ratio of the watery feces excreted among all evacuations in the first predetermined period, and in generation of the alert information, a second watery feces ratio indicating a ratio of the watery feces excreted among all evacuations in the second predetermined period may be calculated, and in a case where the second watery feces ratio is higher than the first watery feces ratio, second alert information indicating that the user is highly likely to have diarrhea may be generated.

According to this configuration, the first watery feces ratio indicating a ratio of watery feces excreted among all evacuations in the first predetermined period is compared with the second watery feces ratio indicating a ratio of watery feces excreted among all evacuations in the second predetermined period. Therefore, the second alert information indicating that the user is highly likely to have diarrhea can be output according to a change in a shape of feces of an individual user.

Further, in the above information processing method, the excretion related data may include evacuation amount data indicating an evacuation amount, the reference database may include a first evacuation amount indicating an average evacuation amount per time in the first predetermined period, and in generation of the alert information, a second evacuation amount indicating an average evacuation amount per time in the second predetermined period may be calculated, and in a case where the second evacuation amount is smaller than the first evacuation amount, third alert information indicating that there is a high possibility that a meal intake amount of the user is insufficient may be generated.

According to this configuration, since the first evacuation amount indicating an average evacuation amount per time in the first predetermined period is compared with the second evacuation amount indicating an average evacuation amount per time in the second predetermined period, it is possible to output the third alert information indicating that there is a high possibility that a meal intake amount of the user is insufficient according to a change in an evacuation amount of an individual user.

Further, in the above information processing method, the excretion related data may include evacuation color data indicating a color of excreted feces, the reference database may include a first evacuation color ratio indicating a ratio of excretion of feces of a predetermined color among all evacuations in the first predetermined period, and in generation of the alert information, a second evacuation color ratio indicating a ratio of excretion of feces of the predetermined color among all evacuations in the second predetermined period may be calculated, and in a case where the first evacuation color ratio and the second evacuation color ratio are different, fourth alert information indicating that a color of feces of the user changes may be generated.

According to this configuration, since the first evacuation color ratio indicating a ratio of excretion of feces of a predetermined color among all evacuations in the first predetermined period is compared with the second evacuation color ratio indicating a ratio of excretion of feces of a predetermined color among all evacuations in the second predetermined period, it is possible to output the fourth alert information indicating that a color of feces of the user changes according to a change in a color of feces of an individual user.

Further, in the above information processing method, the excretion related data may include excrement type data indicating that excrement of the user is urine, the reference database may include a first number of times of urination indicating an average number of times of urination per day in the first predetermined period, and in generation of the alert information, a second number of times of urination indicating an average number of times of urination per day in the second predetermined period may be calculated, and in a case where the second number of times of urination is larger than the first number of times of urination, fifth alert information indicating that the user tends to have frequent urination may be generated.

According to this configuration, since the first number of times of urination indicating an average number of times of urination per day in the first predetermined period is compared with the second number of times of urination indicating an average number of times of urination per day in the second predetermined period, the fifth alert information indicating that the user tends to have frequent urination can be output according to a change in the number of times of urination of an individual user.

Further, in the above information processing method, the excretion related data may include excrement type data indicating that excrement of the user is urine, the reference database may include a first number of times of urination indicating an average number of times of urination per day in the first predetermined period, and in generation of the alert information, a second number of times of urination indicating an average number of times of urination per day in the second predetermined period may be calculated, and in a case where the second number of times of urination is smaller than the first number of times of urination, sixth alert information indicating that there is a high possibility that a water intake amount of the user is insufficient may be generated.

According to this configuration, since the first number of times of urination indicating an average number of times of urination per day in the first predetermined period is compared with the second number of times of urination indicating an average number of times of urination per day in the second predetermined period, the sixth alert information indicating that there is a high possibility that a water intake amount of the user is insufficient can be output according to a change in the number of times of urination of an individual user.

Further, in the above information processing method, the excretion related data may include urine specific gravity data indicating whether or not specific gravity of urine of the user is higher than a predetermined range, the reference database may include a first specific gravity ratio indicating a ratio in which specific gravity of the urine is higher than the predetermined range among all urinations in the first predetermined period, and in generation of the alert information, a second specific gravity ratio indicating a ratio in which specific gravity of the urine is higher than the predetermined range among all urinations in the second predetermined period may be calculated, and in a case where the second specific gravity ratio is higher than the first specific gravity ratio, seventh alert information indicating that the user is highly likely to be dehydrated may be generated.

According to this configuration, the first specific gravity ratio indicating a ratio in which specific gravity of urine among all urinations in the first predetermined period is higher than a predetermined range is compared with the second specific gravity ratio indicating a ratio in which specific gravity of urine among all urinations in the second predetermined period is higher than a predetermined range. Therefore, the seventh alert information indicating that the user is highly likely to be dehydrated can be output according to a change in specific gravity of urine of an individual user.

Further, in the above information processing method, the excretion related data may include bleeding data indicating that the user bleeds at a time of evacuation or urination, the reference database may include a first number of times of bleeding indicating the number of times of bleeding in the first predetermined period, and in generation of the alert information, a second number of times of bleeding indicating the number of times of bleeding in the second predetermined period may be calculated, and in a case where the first number of times of bleeding is smaller than a predetermined number of times and the second number of times of bleeding is equal to or more than a predetermined number of times, eighth alert information indicating that there is a high possibility that the user is bleeding at the time of evacuation or urination may be generated.

According to this configuration, since each of the first number of times of bleeding indicating the number of times of bleeding in the first predetermined period and the second number of times of bleeding indicating the number of times of bleeding in the second predetermined period is compared with a predetermined number of times, it is possible to output the eighth alert information indicating that there is a high possibility that the user is bleeding at the time of evacuation or urination according to a change in the number of times of bleeding of an individual user.

Further, in the above information processing method, the excretion related data may include evacuation related data related to evacuation of the user and urination related data related to urination of the user, and the second predetermined period for acquiring the evacuation related data may be longer than the second predetermined period for acquiring the urination related data.

According to this configuration, since the number of times of urination in one day is generally larger than the number of times of evacuation in one day, a period for acquiring the necessary number of samples is longer for evacuation related data than for urination related data. Since the second predetermined period for acquiring evacuation related data is longer than the second predetermined period for acquiring urination related data, the number of samples necessary for determination can be acquired.

Further, in the above information processing method, in generation of the alert information, related data related to the generated alert information may be further acquired, and in output of the alert information, the related data may be output together with the alert information.

According to this configuration, since related data related to alert information is output together with the alert information, a person who looks at the alert information can analyze a factor for which the alert information is output based on the related data.

Further, in the above-described information processing method, the related data may include a type of medicine taken by the user.

According to this configuration, since a type of medicine taken by the user is output together with alert information, a person who looks at the alert information can analyze a factor for which the alert information is output according to the type of medicine taken by the user.

Further, in the above-described information processing method, the related data may include at least one of meal content of the user, an environment in a house of the user, and an activity amount of the user.

According to this configuration, since at least one of meal content of the user, an environment in a house of the user, and an activity amount of the user is output together with the alert information, a person who looks at the alert information can analyze a factor for which the alert information is output according to at least one of meal content of the user, an environment in a house of the user, and an activity amount of the user.

The present disclosure can be implemented not only as the information processing method that performs characteristic processing as described above, but also as an information processing device or the like having a characteristic configuration corresponding to a characteristic method performed according to the information processing method. Further, the present disclosure can also be implemented as a computer program that causes a computer to execute characteristic processing included in the information processing method described above. Thus, an effect as in the above information processing method can also be achieved by another aspect described below.

An information processing device according to another aspect of the present disclosure includes a first data acquisition part that acquires first data in which excretion related data of a user acquired by a sensor installed in a toilet and a user ID for identifying the user are associated with each other in a first predetermined period, a reference database generation part that generates a reference database indicating an excretion tendency of the user based on the acquired first data, a second data acquisition part that acquires second data in which the excretion related data of the user acquired by the sensor installed in the toilet and the user ID are associated with each other in a second predetermined period, an alert information generation part that generates alert information to the user based on the reference database and the second data, and an output part that outputs the generated alert information.

A non-transitory computer readable recording medium storing an information processing program according to another aspect of the present disclosure causes a computer to function to acquire first data in which excretion related data of a user acquired by a sensor installed in a toilet and a user ID for identifying the user are associated with each other in a first predetermined period, generate a reference database indicating an excretion tendency of the user based on the acquired first data, acquire second data in which the excretion related data of the user acquired by the sensor installed in the toilet and the user ID are associated with each other in a second predetermined period, generate alert information to the user based on the reference database and the second data, and output the generated alert information.

An embodiment of the present disclosure will be described below with reference to the accompanying drawings. Note that the embodiment below is an example of embodiment of the present disclosure, and is not intended to limit the technical scope of the present disclosure.

### EMBODIMENT

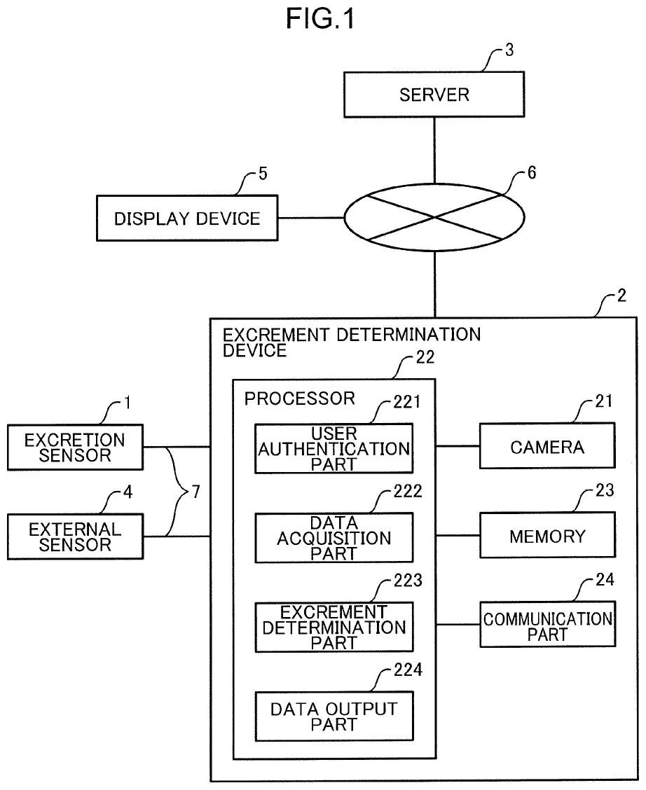

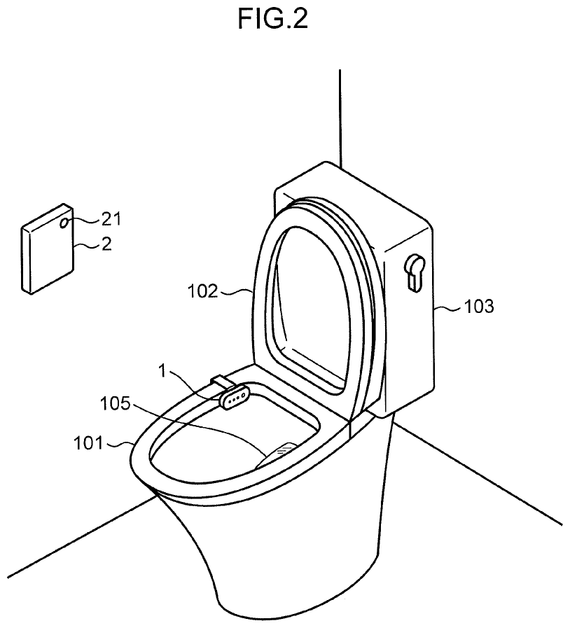

FIG. 1 is a diagram illustrating a configuration of an excretion management system in an embodiment of the present disclosure. FIG. 2 is a diagram for explaining arrangement positions of an excretion sensor 1 and an excrement determination device 2 in the embodiment of the present disclosure.

The excretion management system is a system introduced into a facility such as a nursing home and a hospital, manages a state of excrement of the user who uses the facility, and presents various pieces of alert information to an administrator. The user is, for example, a care receiver who receives care in a nursing home and a patient who receives treatment in a hospital. The administrator is, for example, a caregiver for a care receiver, a care manager, a doctor in charge of patient treatment, a nurse, or the like.

The excretion management system illustrated in FIG. 1 includes the excretion sensor 1, the excrement determination device 2, a server 3, an external sensor 4, and a display device 5.

The excrement determination device 2, the server 3, and the display device 5 are communicably connected to each other via a network 6. The network 6 is, for example, a wide-area communication network including an Internet communication network and a mobile phone communication network. The excrement determination device 2 is installed, for example, on a wall surface in a toilet. The excretion sensor 1 is communicably connected to the excrement determination device 2 via a communication path 7. The communication path 7 is a communication path of near field communication such as Bluetooth (registered trademark), infrared communication, and near field communication (NFC). Note that the communication path 7 may be a wired communication path. Further, the external sensor 4 is communicably connected to the excrement determination device 2 via the communication path 7.

The excretion sensor 1 is arranged in a toilet bowl 101. As illustrated in FIG. 2, the excretion sensor 1 is hung on an edge of an opening formed in an upper part of the toilet bowl 101 that receives feces and urine.

A water reservoir portion 105 is provided at a bottom portion of the toilet bowl 101. The water reservoir portion 105 is connected to a drain passage (not shown) for discharging feces and urine from the inside of the toilet bowl 101. Feces and urine excreted into the toilet bowl 101 are caused to flow through the drain passage. An upper part of the toilet bowl 101 is provided with a toilet seat 102 on which the user sits. The toilet seat 102 rotates up and down. The user sits down in a state where the toilet seat 102 is lowered onto the toilet bowl 101. At the rear of the toilet bowl 101, a flush tank 103 that stores water for flushing feces and urine is provided.

The excretion sensor 1 includes a camera. The camera is installed on the toilet bowl 101 so that the inside of the toilet bowl 101 can be photographed. The camera is, for example, a high sensitivity and wide angle camera capable of capturing a color image having three color components of a red (R) component, a green (G) component, and a blue (B) component. However, this is an example, and size, sensitivity, and the number of elements of image detection elements are not defined as performance of the camera. Specifically, the camera is configured by a high sensitivity camera having a ¼ inch CMOS. The camera includes a wide angle camera having an angle of view in a horizontal direction of 120 degrees and an angle of view in a vertical direction of 100 degrees. Note that these numerical values for inch and angle of view are merely examples, and other values may be employed. The camera photographs the inside of the toilet bowl 101 at a predetermined frame rate, includes time information indicating photographing time in obtained image data, and transmits image data including time information to the excrement determination device 2. The time information is information including year, month, day, hour, minute, and second such as 15:00:00 on Apr. 1, 2021. However, this is an example, and the time information may be information including hour, minute, and second.

The external sensor 4 includes an environment sensor that measures temperature and humidity of a room in which the user mainly lives, an opening and closing sensor that detects opening and closing of a door of a refrigerator, and an activity meter that measures an activity amount of the user, such as a pedometer. The external sensor 4 transmits sensing data to the excrement determination device 2.

The excrement determination device 2 includes a camera 21, a processor 22, a memory 23, and a communication part 24.

The camera 21 captures the user's face. The camera 21 is arranged at a position where a face of the user sitting on the toilet seat 102 can be captured.

The memory 23 is a storage device capable of storing various types of information, such as a random access memory (RAM), a solid state drive (SSD), or a flash memory. The memory 23 stores a face image of the user and a user ID for identifying the user in association with each other.

The communication part 24 receives image data obtained by photographing the inside of the toilet bowl 101 transmitted by the excretion sensor 1.

The processor 22 is, for example, a central processing unit (CPU). A user authentication part 221, a data acquisition part 222, an excrement determination part 223, and a data output part 224 are realized by the processor 22.

The user authentication part 221 acquires a user ID associated with a face image captured by the camera 21 from the memory 23.

Note that the camera 21 may capture a barcode or a two dimensional code in which a user ID is stored. The user authentication part 221 may read a user ID from a barcode or a two dimensional code captured by the camera 21.

Further, the excrement determination device 2 may include an IC card reader instead of the camera 21. The IC card reader reads information stored in an IC card in a contactless manner by NFC. An IC card stores in advance a user ID for identifying the user. The user brings an IC card in which a user ID of the user is stored close to the IC card reader. By the above, the IC card reader acquires the user ID from the nearby IC card. The user authentication part 221 acquires the user ID from the IC card reader.

Further, the excrement determination device 2 may include a microphone instead of the camera 21. The user authentication part 221 may acquire voice of the user with the microphone and acquire a user ID associated with the acquired voice from the memory 23.

The data acquisition part 222 acquires image data captured by the excretion sensor 1. The data acquisition part 222 acquires image data from the communication part 24.

The excrement determination part 223 determines a type of excrement indicating whether the excrement is feces or urine, a shape of excreted feces, an amount of excreted feces, a color of excreted feces, an amount of excreted urine, specific gravity of excreted urine, and bleeding by performing image recognition on image data acquired by the data acquisition part 222.

The type of excrement indicates whether excrement is feces or urine. Here, determination of the type of excrement by the excrement determination part 223 will be described.

First, the excrement determination part 223 calculates a difference between image data acquired by the data acquisition part 222 and reference image data stored in the memory 23. The reference image data is generated based on, for example, a plurality of pieces of color image data obtained by the excretion sensor 1 capturing, a plurality of times, a state of the inside of the toilet bowl 101 where evacuation or urination is not performed. That is, the reference image data is color image data in a default state in the toilet bowl 101 in which evacuation or urination is not performed. Therefore, image data indicating evacuation or urination is extracted as a difference between image data captured at the time of evacuation or urination and reference image data is calculated.

Next, the excrement determination part 223 calculates an RGB ratio of an R component, a G component, and a B component of each pixel included in calculated difference image data. Next, the excrement determination part 223 calculates the total number of pixels in which the calculated RGB ratio falls within a predetermined evacuation reference ratio range. Here, the RGB ratio is, for example, a ratio of a luminance value of an R component, a luminance value of a G component, and a luminance value of a B component of each pixel in difference image data. The evacuation reference ratio range is a range of RGB ratios of typical evacuation calculated by analysis of a plurality of pieces of image data including various evacuation images. Finally, if the total number of pixels whose calculated RGB ratio falls within the predetermined evacuation reference ratio range is equal to or more than a threshold, the excrement determination part 223 determines that image data captured by the excretion sensor 1 includes an evacuation image and excrement is feces.

Note that the excrement determination part 223 may determine whether or not a urination image is also included in image data by processing similar to that for an evacuation image. That is, the excrement determination part 223 calculates the total number of pixels in which an RGB ratio of each pixel included in a difference image falls within a predetermined urination reference ratio range. When the total number of pixels in which a calculated RGB ratio falls within a predetermined urination reference ratio range is equal to or more than a threshold, the excrement determination part 223 may determine that image data captured by the excretion sensor 1 includes a urination image and excrement is urine.

A shape of excreted feces indicates whether excreted feces are hard feces, normal feces, or watery feces. In a case where excrement is feces, the excrement determination part 223 performs image recognition on image data acquired by the data acquisition part 222 to determine whether excreted feces are hard feces, normal feces, or watery feces.

An amount of excreted feces indicates an area of excreted feces. In a case where excrement is feces, the excrement determination part 223 determines an area of excreted feces as an amount of excreted feces by performing image recognition on image data acquired by the data acquisition part 222.

A color of excreted feces indicates whether a color of excreted feces is black, white, or green. In a case where excrement is feces, the excrement determination part 223 performs image recognition on image data acquired by the data acquisition part 222 to determine whether a color of the excreted feces is black, white, or green. A color of feces may change depending on a type of medicine taken by the user. For example, black feces are feces that may be excreted in a case where sodium ferrous citrate or the like is taken. For example, the excrement determination part 223 calculates a G/R value and a B/R value based on an R value, a G value, and a B value included in image data. Specifically, the excrement determination part 223 sets a detection area in image data acquired by the data acquisition part 222, and calculates a G/R value and a B/R value for each piece of a plurality of pixel data constituting a detection area. The G/R value is a value obtained by dividing the G value by the R value and expressed in %. The B/R value is a value obtained by dividing the B value by the R value and expressed in %. The R value is a gradation value of an R (red) component of pixel data, the G value is a gradation value of a G (green) component of pixel data, and the B value is a gradation value of a B (blue) component of pixel data. The R value, the G value, and the B value are, for example, 8-bit (0 to 255) values. However, this is an example, and the R value, the G value, and the B value may be expressed by other bit numbers. Further, the detection area is a rectangular region including the water reservoir portion 105 of the toilet bowl 101.

When each of the calculated G/R value and B/R value and each of the R value, the G value, and the B value included in image data satisfy a predetermined black feces condition, the excrement determination part 223 determines that image data includes an image of black feces. For example, the black feces condition is a condition that the G/R value is within a first range, the B/R value is within a second range, and each of the R value, the G value, and the B value is within a third range.

The black feces condition is a condition that the G/R value is D1% or more and D2% or less, the B/R value is D3% or more and D4% or less, and each of the R value, the G value, and the B value is zero or more and E or less. For example, D3%, D4% may be D3%<D1%, D4%=D2%. Note that, in a case where image data is 8 bits, since each of the R value, the G value, and the B value takes a value of 0 to 255, E is 0 or more and 255 or less. Specifically, D1 is, for example, 85 or more and 95 or less, and preferably 88 or more and 92 or less. D2 is, for example, 105 or more and 115 or less, and preferably 108 or more and 112 or less. D3 is, for example, 55 or more and 65 or less, and preferably 58 or more and 62 or less. D4 is, for example, 105 or more and 115 or less, and preferably 108 or more and 112 or less. In a case where image data is 8 bits, E is, for example, 85 or more and 95 or less, and preferably 88 or more and 92 or less. In a case where a bit number of image data is optional, E is, for example, 33% or more and 37% or less, and preferably 34% or more and 36% or less.

The excrement determination part 223 determines that a color of excreted feces is black in a case where there are a predetermined number of pixels or more of pixel data satisfying a black feces condition in image data. The predetermined number of pixels is a preset number of pixels indicating that pixel data satisfying a black feces condition is not noise but pixel data of black feces.

Similarly, each of the calculated G/R value and B/R value and each of the R value, the G value, and the B value included in image data satisfy a predetermined white feces condition, the excrement determination part 223 may determine that image data includes an image of white feces. Further, each of the calculated G/R value and B/R value and each of the R value, the G value, and the B value included in image data satisfy a predetermined green feces condition, the excrement determination part 223 may determine that image data includes an image of green feces.

An amount of excreted urine indicates an area of excreted urine. In a case where excrement is urine, the excrement determination part 223 determines an area of excreted urine as an amount of excreted urine by performing image recognition on image data acquired by the data acquisition part 222.

Specific gravity of excreted urine indicates whether or not specific gravity of excreted urine is greater than a predetermined range. The predetermined range is a range of a general normal value, for example, a range of 1.010 to 1.030. When specific gravity of excreted urine is within a normal value range, urine diffuses into the water reservoir portion 105 without accumulating at the bottom of the water reservoir portion 105. For this reason, the number of pixels representing urine increases from start of urination to end of urination, and is maintained at a high numerical value even after the end of urination. On the other hand, in a case where specific gravity of excreted urine is larger than a normal value range, urine sinks to the bottom of the water reservoir portion 105. For this reason, the number of pixels representing urine increases from start of urination to end of urination, and decreases after the end of urination. In a case where the number of pixels representing urine in a detection area after end of urination is smaller than a threshold, the excrement determination part 223 determines that specific gravity of excreted urine is larger than a normal value range. The detection area is a rectangular region including the water reservoir portion 105 of the toilet bowl 101.

Determination of bleeding indicates whether or not excrement contains blood. The excrement determination part 223 extracts a blood image indicating blood from a plurality of images acquired by the data acquisition part 222. Here, the excrement determination part 223 may extract, as a blood image, a region where a predetermined number or more of pixels having a predetermined RGB value indicating blood are continuous in a specific area of a plurality of images. The “pixels having a predetermined RGB value indicating blood” refer to, for example, pixels having an RGB value within a predetermined range with respect to a predetermined RGB value indicating blood. The predetermined range refers to a predetermined range of RGB values that can be regarded as blood. Note that in an external injury such as hemorrhoids, blood is distributed in a dot shape. For this reason, a plurality of blood images may be extracted in one image.

Note that in a case where an evacuation image or a urination image is extracted, the excrement determination part 223 may determine an excretion start timing based on time information corresponding to the extracted evacuation image or urination image. The excretion start timing refers to time information included in an image from which an evacuation image or a urination image is extracted. Further, in a case where a blood image is extracted, the excrement determination part 223 may determine a bleeding start timing on the basis of time information corresponding to an extracted blood image and determine blood size indicating size of blood on the basis of a blood image. The bleeding start timing refers to time information included in an image from which a blood image is extracted.

The data output part 224 generates excretion related data related to excretion of the user on the basis of a determination result by the excrement determination part 223. The excretion related data includes a time stamp including excretion start date and time and excretion end date and time, excrement type data indicating whether excrement is feces or urine, feces shape data indicating whether a shape of excreted feces is hard feces, normal feces, or watery feces, evacuation amount data indicating an evacuation amount, evacuation color data indicating a color of excreted feces, urination amount data indicating a urination amount, urine specific gravity data indicating whether or not specific gravity of urine of the user is higher than a predetermined range, and bleeding data indicating that the user bleeds at the time of evacuation or urination.

Further, the time stamp, the excrement type data indicating that excrement is feces, the feces shape data, the evacuation amount data, the evacuation color data, and the bleeding data are evacuation related data related to evacuation of the user. Further, the time stamp, the excrement type data indicating that excrement is urine, the urination amount data, the urine specific gravity data, and the bleeding data are urination related data related to urination of the user.

The data output part 224 assigns a user ID to the excretion related data, and outputs the excretion related data to which a user ID is assigned to the communication part 24. The communication part 24 transmits data in which the excretion related data and the user ID are associated with each other to the server 3.

Further, the data output part 224 adds a user ID and a time stamp to sensing data received from the external sensor 4, and outputs the sensing data to which the user ID and the time stamp are added to the communication part 24. The sensing data includes environment information indicating temperature and humidity of a room in which the user mainly lives, refrigerator opening and closing information indicating opening and closing of a door of a refrigerator, and activity amount information indicating an activity amount of the user. The time stamp includes measurement date and time of sensing data or receiving date and time of sensing data. The communication part 24 transmits sensing data to which a user ID and a time stamp are added to the server 3.

The server 3 is a cloud server including one or more computers, for example.

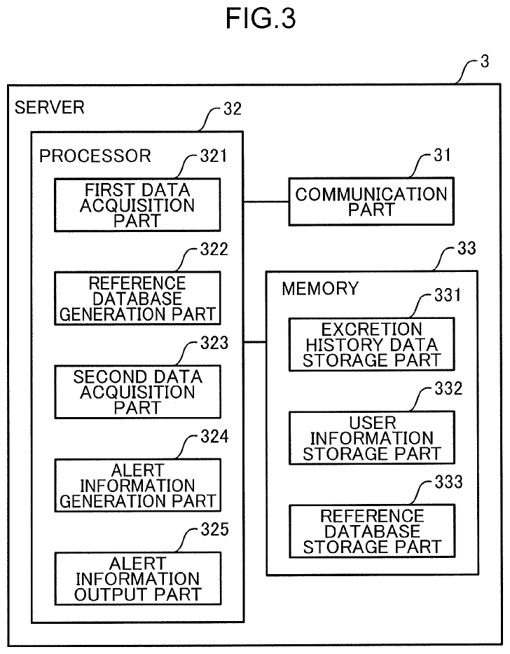

FIG. 3 is a block diagram illustrating an example of a configuration of the server 3 according to the embodiment of the present disclosure.

The server 3 illustrated in FIG. 3 includes a communication part 31, a processor 32, and a memory 33.

The communication part 31 receives data in which excretion related data transmitted by the excrement determination device 2 is associated with a user ID.

The processor 32 is, for example, a CPU. The processor 32 realizes a first data acquisition part 321, a reference database generation part 322, a second data acquisition part 323, an alert information generation part 324, and an alert information output part 325.

The memory 33 is, for example, a storage device capable of storing various types of information such as a RAM, an SSD, or a flash memory. An excretion history data storage part 331, a user information storage part 332, and a reference database storage part 333 are realized by the memory 33.

In a first predetermined period, the first data acquisition part 321 acquires first data in which excretion related data of the user acquired by the excretion sensor 1 installed in a toilet is associated with a user ID for identifying the user. The first predetermined period is one month, for example. In the first predetermined period, the first data acquisition part 321 acquires first data in which excretion related data and a user ID are associated with each other from the excrement determination device 2, and stores the acquired first data in the excretion history data storage part 331.

The excretion history data storage part 331 is a database that stores data in which excretion related data and a user ID are associated with each other as excretion history data.

The user information storage part 332 is a database that stores user information including a user ID, medicine taking information, meal information, environment information, refrigerator opening and closing information, and activity amount information. The medicine taking information includes a type of medicine taken by the user and medicine taking date and time. The meal information includes content of a meal that the user eats and meal date and time. The environment information includes temperature and humidity of a room in which the user mainly lives. The refrigerator opening and closing information includes the number of times a door of a refrigerator is opened and closed in one day. The activity amount information includes an activity amount such as the number of steps of the user in one day.

The communication part 31 receives medicine taking information and meal information transmitted by another personal computer (not illustrated). The processor 32 stores medicine taking information and meal information received by the communication part 31 in the user information storage part 332. Another personal computer receives input of medicine taking information and meal information by a caregiver, a nurse, or the like, and transmits the input medicine taking information and meal information to the server 3. Further, the communication part 31 receives sensing data transmitted by the excrement determination device 2. The processor 32 stores environment information, refrigerator opening and closing information, and activity amount information in the user information storage part 332 based on sensing data received by the communication part 31.

The reference database generation part 322 generates a reference database indicating an excretion tendency of the user on the basis of first data acquired in the first predetermined period by the first data acquisition part 321. The reference database generation part 322 reads first data acquired in the first predetermined period from the excretion history data storage part 331, and generates a reference database on the basis of the read first data.

The reference database generation part 322 uses feces shape data in the first predetermined period to calculate a first hard feces ratio indicating a ratio of hard feces excreted among all evacuations in the first predetermined period. Further, the reference database generation part 322 uses feces shape data in the first predetermined period to calculate a first watery feces ratio indicating a ratio of watery feces excreted among all evacuations in the first predetermined period. Further, the reference database generation part 322 calculates a first evacuation amount indicating an average evacuation amount per time in the first predetermined period by using evacuation amount data in the first predetermined period.

Further, the reference database generation part 322 uses evacuation color data in the first predetermined period to calculate a first evacuation color ratio indicating a ratio of feces of a predetermined color excreted among all evacuations in the first predetermined period. The predetermined color is, for example, black, white, or green. Further, the reference database generation part 322 calculates a first number of times of urination indicating an average number of times of urination per day in the first predetermined period using excrement type data in the first predetermined period. Further, the reference database generation part 322 uses urine specific gravity data in the first predetermined period to calculate a first specific gravity ratio indicating a ratio in which specific gravity of urine is higher than a predetermined range among all urinations in the first predetermined period. Further, the reference database generation part 322 calculates a first number of times of bleeding indicating the number of times of bleeding in the first predetermined period using bleeding data in the first predetermined period.

The reference database generation part 322 generates a reference database including the first hard feces ratio, the first watery feces ratio, the first evacuation amount, the first evacuation color ratio, the first number of times of urination, the first specific gravity ratio, and the first number of times of bleeding, and stores the generated reference database in the reference database storage part 333 in association with a user ID.

The reference database storage part 333 stores a reference database generated by the reference database generation part 322.

In a second predetermined period, the second data acquisition part 323 acquires second data in which excretion related data of the user acquired by the excretion sensor 1 installed in a toilet is associated with a user ID. The second predetermined period is shorter than the first predetermined period. The second predetermined period is, for example, two weeks, one week, or three days. In the second predetermined period, the second data acquisition part 323 acquires second data in which excretion related data and a user ID are associated with each other from the excrement determination device 2, and stores the acquired second data in the excretion history data storage part 331.

Note that the excretion related data includes evacuation related data related to evacuation of the user and urination related data related to urination of the user. The second predetermined period for acquiring the evacuation related data is, for example, two weeks, and the second predetermined period for acquiring the urination related data is, for example, one week. Generally, evacuation is performed one to three times a day, and urination is performed seven to eight times a day. As described above, the number of samples required to determine alert information is different between evacuation and urination. For this reason, the second predetermined period for acquiring the evacuation related data is preferably longer than the second predetermined period for acquiring the urination related data. Further, evacuation is easily affected by dietary intake or physical condition (such as exercise or constipation), and thus long-term observation is necessary. On the other hand, since dehydration or cystitis is rapidly deteriorated, it is preferable to confirm urination in a short period of time depending on an excretion situation. However, even in a case of urination, it is preferable to check a long-term tendency change in heart failure or the like. Further, the second predetermined period may be determined according to alert information to be generated.

The alert information generation part 324 generates alert information to the user based on a reference database stored in the reference database storage part 333 and second data stored in the excretion history data storage part 331.

The alert information generation part 324 calculates a second hard feces ratio indicating a ratio of hard feces excreted among all evacuations in the second predetermined period using feces shape data in the second predetermined period. The second predetermined period is, for example, two weeks. Then, in a case where a second hard feces ratio is larger than a first hard feces ratio included in a reference database, the alert information generation part 324 generates first alert information indicating that the user is highly likely to have constipation.

Further, the alert information generation part 324 calculates a second watery feces ratio indicating a ratio of excretion of watery feces among all evacuations in the second predetermined period using feces shape data in the second predetermined period. The second predetermined period is, for example, two weeks. Then, in a case where a second watery feces ratio is larger than a first watery feces ratio included in a reference database, the alert information generation part 324 generates second alert information indicating that the user is highly likely to have diarrhea.

Further, the alert information generation part 324 calculates a second evacuation amount indicating an average evacuation amount per time in the second predetermined period using evacuation amount data in the second predetermined period. The second predetermined period is, for example, two weeks. Then, in a case where a second evacuation amount is smaller than a first evacuation amount included in a reference database, the alert information generation part 324 generates third alert information indicating that there is a high possibility that a meal intake amount of the user is insufficient. Note that the alert information generation part 324 may generate third alert information in a case where a second evacuation amount is decreased by a predetermined ratio from a first evacuation amount. The predetermined ratio is, for example, 20%. The predetermined ratio may be manually adjusted by an administrator depending on a change in a medical history and a state of the user (for example, wearing of a diaper).

Further, the alert information generation part 324 calculates a second evacuation color ratio indicating a ratio of excretion of feces of a predetermined color among all evacuations in the second predetermined period using evacuation color data in the second predetermined period. The second predetermined period is, for example, two weeks. Then, in a case where a first evacuation color ratio and a second evacuation color ratio included in a reference database are different, the alert information generation part 324 generates fourth alert information indicating that a color of feces of the user changes. Note that the alert information generation part 324 may calculate a second evacuation color ratio for each color of feces, and may determine whether or not a first evacuation color ratio and a second evacuation color ratio are different for each color of feces.

Further, the alert information generation part 324 calculates a second number of times of urination indicating an average number of times of urination per day in the second predetermined period using excrement type data in the second predetermined period. The second predetermined period is one week, for example. Then, in a case where the second number of times of urination is larger than a first number of times of urination included in a reference database, the alert information generation part 324 generates fifth alert information indicating that the user tends to have frequent urination.

Note that the alert information generation part 324 may generate the fifth alert information in a case where a second number of times of urination is increased by a predetermined ratio from a first number of times of urination. The predetermined ratio is, for example, 20%. The predetermined ratio may be manually adjusted by an administrator depending on a change in a medical history and a state of the user (for example, wearing of a diaper).

Further, the reference database generation part 322 may calculate a first urination small amount ratio indicating a ratio in which a urination amount is smaller than a predetermined amount among all urinations in the first predetermined period using urination amount data in the first predetermined period. The alert information generation part 324 may calculate a second urination small amount ratio indicating a ratio in which a urination amount is smaller than a predetermined amount among all urinations in the second predetermined period using urination amount data in the second predetermined period. Then, the alert information generation part 324 may generate the fifth alert information in a case where a second number of times of urination is larger than a first number of times of urination and a second small urination amount ratio is larger than a first small urination amount ratio. Note that the predetermined amount may be manually adjusted by an administrator according to a medical history and a change in a state of the user (for example, wearing of a diaper), and a season.

Further, the reference database generation part 322 may calculate a first number of times of urination at night indicating an average number of times of urination at night per day during the first predetermined period. The alert information generation part 324 may calculate a second number of times of urination at night indicating an average number of times of urination at night per day during the second predetermined period. Then, in a case where the second number of times of urination at night is larger than the first number of times of urination at night included in a reference database, the alert information generation part 324 may generate alert information indicating that the user tends to have frequent urination at night.

Further, the alert information generation part 324 may generate alert information indicating that the user tends to have frequent urination at night in a case where a second number of times of urination at night increases by a predetermined ratio from a first number of times of urination at night. The predetermined ratio is, for example, 20%. The predetermined ratio may be manually adjusted by an administrator depending on a change in a medical history and a state of the user (for example, wearing of a diaper). Further, the alert information generation part 324 may generate alert information indicating that the user tends to have frequent urination at night in a case where a second number of times of urination at night is larger than a first number of times of urination at night and a second urination small amount ratio is larger than a first urination small amount ratio.

Further, the alert information generation part 324 calculates a second number of times of urination indicating an average number of times of urination per day in the second predetermined period using excrement type data in the second predetermined period. The second predetermined period is one week, for example. Then, in a case where a second number of times of urination is smaller than a first number of times of urination included in a reference database, the alert information generation part 324 generates sixth alert information indicating that there is a high possibility that a water intake amount of the user is insufficient.

Note that the alert information generation part 324 may generate the sixth alert information in a case where a second number of times of urination is decreased by a predetermined ratio from a first number of times of urination. The predetermined ratio is, for example, 20%. The predetermined ratio may be manually adjusted by an administrator depending on a change in a medical history and a state of the user (for example, wearing of a diaper).

Further, the reference database generation part 322 may calculate a first urination small amount ratio indicating a ratio in which a urination amount is smaller than a predetermined amount among all urinations in the first predetermined period using urination amount data in the first predetermined period. The alert information generation part 324 may calculate a second urination small amount ratio indicating a ratio in which a urination amount is smaller than a predetermined amount among all urinations in the second predetermined period using urination amount data in the second predetermined period. Then, the alert information generation part 324 may generate the sixth alert information in a case where a second number of times of urination is smaller than a first number of times of urination and a second small urination amount ratio is larger than a first small urination amount ratio. The predetermined amount may be manually adjusted by an administrator according to a medical history and a change in a state of the user (for example, wearing of a diaper), and a season.

Further, the alert information generation part 324 calculates a second specific gravity ratio indicating a ratio in which specific gravity of urine is higher than a predetermined range among all urinations in the second predetermined period, using urine specific gravity data in the second predetermined period. The second predetermined period is, for example, most recent three days. Then, in a case where a second specific gravity ratio is larger than a first specific gravity ratio included in a reference database, the alert information generation part 324 generates seventh alert information indicating that the user is highly likely to be dehydrated. Dehydration may occur due to imbalance of daily water intake, sweating, and excretion. Since dehydration causes a physically dangerous state, the second predetermined period for generating the seventh alert information is preferably shorter than the second predetermined period for generating the fifth alert information and the sixth alert information.

Note that the alert information generation part 324 may generate the seventh alert information in a case where a second specific gravity ratio increases by a predetermined ratio from a first specific gravity ratio. The predetermined ratio is, for example, 20%. The predetermined ratio may be manually adjusted by an administrator depending on a change in a medical history and a state of the user (for example, wearing of a diaper).

Further, the alert information generation part 324 calculates a second number of times of bleeding indicating the number of times of bleeding in the second predetermined period using bleeding data in the second predetermined period. The second predetermined period is, for example, most recent three days. Then, in a case where a first number of times of bleeding included in a reference database is smaller than a predetermined number of times and a second number of times of bleeding is equal to or more than a predetermined number of times, the alert information generation part 324 generates eighth alert information indicating that there is a high possibility that the user is bleeding at the time of evacuation or urination.

Note that, in the present embodiment, in a case where a first number of times of bleeding included in a reference database is zero and a second number of times of bleeding is one or more, the alert information generation part 324 generates eighth alert information indicating that there is a high possibility that the user is bleeding at the time of evacuation or urination.

Further, the reference database generation part 322 may calculate a first bleeding ratio indicating a ratio of bleeding among all evacuations and urinations in the first predetermined period using bleeding data in the first predetermined period. The alert information generation part 324 may calculate a second bleeding ratio indicating a ratio of bleeding among all evacuations and urinations in the second predetermined period using bleeding data in the second predetermined period. Then, in a case where a second bleeding ratio is larger than a first bleeding ratio included in a reference database, the alert information generation part 324 may generate the eighth alert information.

The alert information output part 325 outputs alert information generated by the alert information generation part 324. The alert information output part 325 outputs generated alert information among the first alert information to the eighth alert information to the display device 5. The communication part 31 transmits alert information output by the alert information output part 325 to the display device 5.

Note that the alert information generation part 324 may further acquire related data related to generated alert information. The alert information output part 325 may output related data together with alert information. The alert information generation part 324 acquires related data related to alert information from the user information storage part 332. For example, the related data includes a type of medicine taken by the user. In a case of generating the first alert information, the second alert information, the fourth alert information, or the fifth alert information, the alert information generation part 324 may acquire a type of medicine taken by the user as related data. Note that acquired related data is determined in advance for alert information.

Further, for example, related data may include at least one of meal information of the user, environment information in a house of the user, and activity amount information of the user. In a case of generating the third alert information, the sixth alert information, or the seventh alert information, the alert information generation part 324 may acquire at least one of meal information of the user, environment information in a house of the user, and activity amount information of the user as related data.

The display device 5 is, for example, a computer owned by an administrator. The display device 5 may be, for example, a stationary computer, a smartphone, or a tablet computer.

The display device 5 includes a communication part, a processor, a memory, a display, and an operation part. The communication part connects the display device 5 to the network 6. The communication part receives alert information from the server 3. The processor includes, for example, a CPU, acquires alert information transmitted by the server 3 using the communication part, generates a display image on the basis of acquired alert information, and displays generated display image on the display. By the above, an administrator can check alert information to the user. The memory includes, for example, a nonvolatile rewritable storage device such as a flash memory. The display includes a display device such as a liquid crystal display panel and an organic EL panel. The operation part includes a keyboard, a mouse, a touch panel, and the like, and receives an instruction from an administrator.

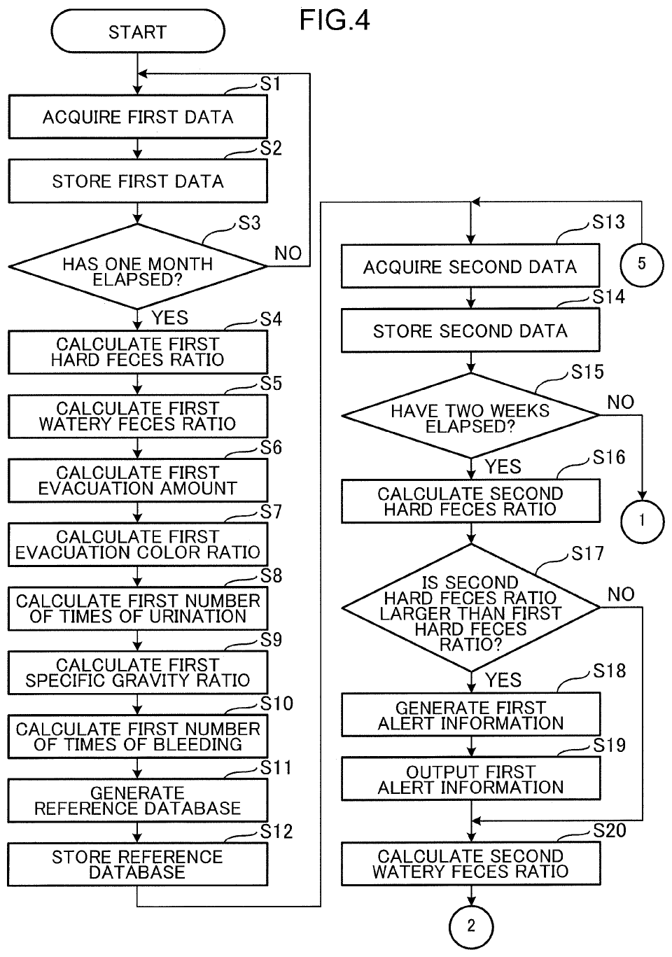

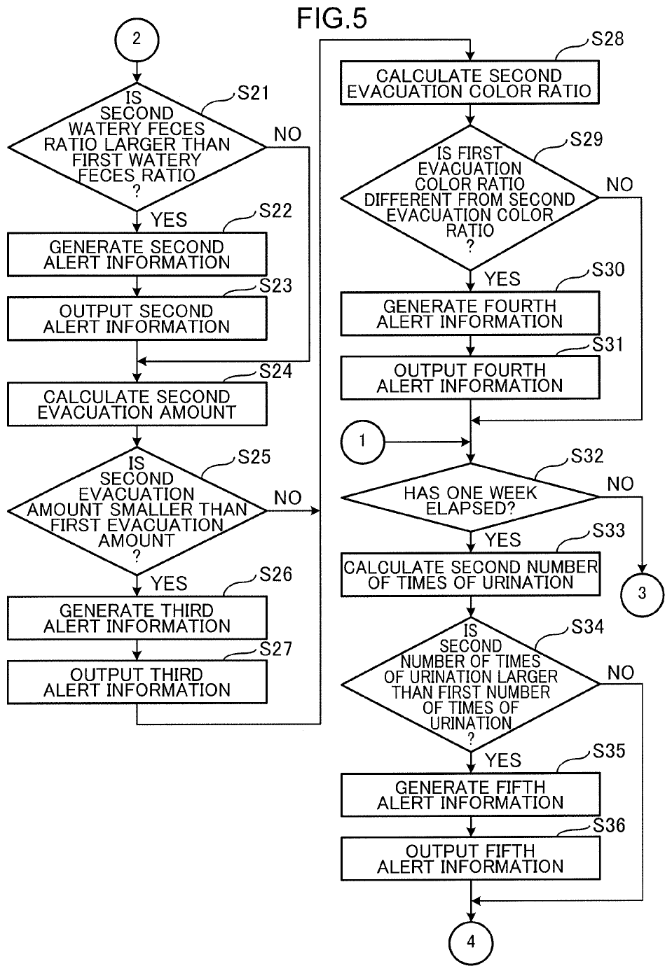

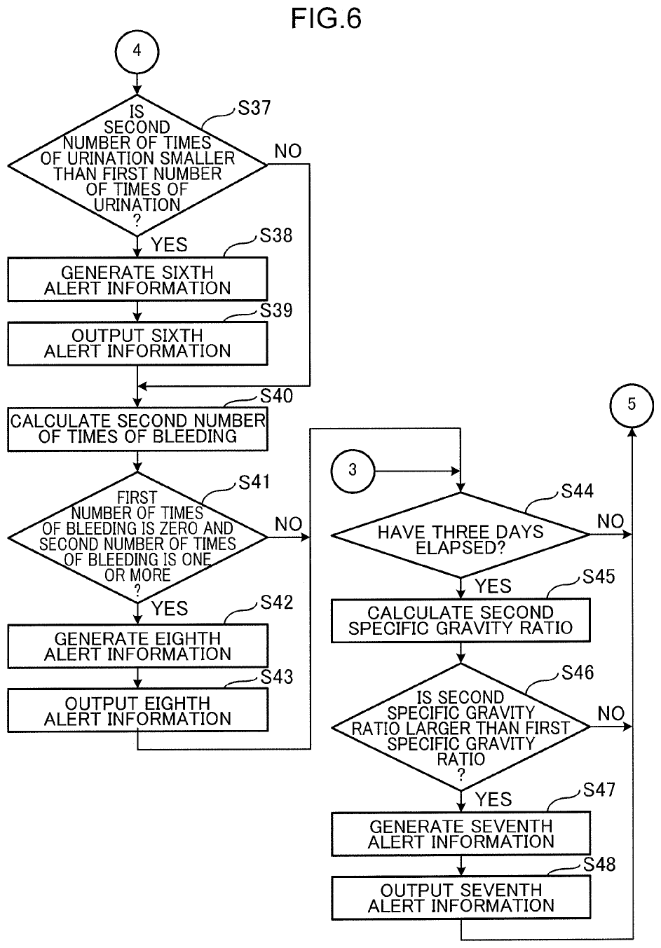

The above is a configuration of the excretion management system. Next, processing of the excretion management system will be described. FIG. 4 is a first flowchart illustrating an example of processing of the server 3 according to the embodiment of the present disclosure, FIG. 5 is a second flowchart illustrating an example of processing of the server 3 according to the embodiment of the present disclosure, and FIG. 6 is a third flowchart illustrating an example of processing of the server 3 according to the embodiment of the present disclosure.

First, in Step S1, the first data acquisition part 321 acquires first data in which excretion related data and a user ID are associated with each other. The first data is acquired every time the user evacuates or urinates.

Next, in Step S2, the first data acquisition part 321 stores the acquired first data in the excretion history data storage part 331.

Next, in Step S3, the first data acquisition part 321 determines whether or not one month has elapsed from start of acquisition of the first data. Here, in a case where it is determined that one month has not elapsed from start of the acquisition of the first data (NO in Step S3), the processing returns to Step S1.

On the other hand, in a case where it is determined that one month has elapsed from start of the acquisition of the first data (YES in Step S3), in Step S4, the reference database generation part 322 calculates a first hard feces ratio indicating a ratio of hard feces excreted in one month by using feces shape data acquired in one month.

Next, in Step S5, the reference database generation part 322 calculates a first watery feces ratio indicating a ratio of watery feces excreted in one month using feces shape data acquired in one month.

Next, in Step S6, the reference database generation part 322 calculates a first evacuation amount indicating an average evacuation amount per time in one month using evacuation amount data acquired in one month.

Next, in Step S7, the reference database generation part 322 calculates a first evacuation color ratio indicating a ratio of excretion of feces of a predetermined color in one month using evacuation color data acquired in one month. The predetermined color is, for example, black.

Next, in Step S8, the reference database generation part 322 calculates a first number of times of urination indicating an average number of times of urination per day in one month using excrement type data acquired in one month.

Next, in Step S9, the reference database generation part 322 calculates a first specific gravity ratio indicating a ratio at which specific gravity of urine in one month is higher than a predetermined range, using urine specific gravity data acquired in one month.

Next, in Step S10, the reference database generation part 322 calculates a first number of times of bleeding indicating the number of times of bleeding in one month using bleeding data acquired in one month.

Next, in Step S11, the reference database generation part 322 generates a reference database including the first hard feces ratio, the first watery feces ratio, the first evacuation amount, the first evacuation color ratio, the first number of times of urination, the first specific gravity ratio, and the first number of times of bleeding.

Next, in Step S12, the reference database generation part 322 stores the generated reference database in the reference database storage part 333 in association with a user ID.

Next, in Step S13, the second data acquisition part 323 acquires second data in which excretion related data and the user ID are associated with each other.

Next, in Step S14, the second data acquisition part 323 stores the acquired second data in the excretion history data storage part 331.

Next, in Step S15, the second data acquisition part 323 determines whether or not two weeks have elapsed from a time point at which it is determined that two weeks have elapsed last time. Here, in a case where it is determined that two weeks have not elapsed from a time point at which it is determined that two weeks have elapsed last time (NO in Step S15), the processing proceeds to Step S32.

On the other hand, in a case where it is determined that two weeks have elapsed from a time point at which it is determined that two weeks have elapsed last time (YES in Step S15), in Step S16, the alert information generation part 324 calculates a second hard feces ratio indicating a ratio of hard feces excreted in two weeks, using feces shape data acquired in two weeks.

Next, in Step S17, the alert information generation part 324 determines whether or not the second hard feces ratio is larger than the first hard feces ratio included in the reference database. Here, in a case where it is determined that the second hard feces ratio is equal to or less than the first hard feces ratio (NO in Step S17), the processing proceeds to Step S20.

On the other hand, in a case where it is determined that the second hard feces ratio is larger than the first hard feces ratio (YES in Step S17), in Step S18, the alert information generation part 324 generates first alert information indicating that the user is highly likely to have constipation.

Next, in Step S19, the alert information output part 325 outputs the first alert information generated by the alert information generation part 324 to the display device 5. The communication part 31 transmits the first alert information output by the alert information output part 325 to the display device 5. The display device 5 receives the first alert information and displays the received first alert information.

Next, in Step S20, the alert information generation part 324 calculates a second watery feces ratio indicating a ratio of watery feces excreted in two weeks by using feces shape data acquired in two weeks.

Next, in Step S21, the alert information generation part 324 determines whether or not the second watery feces ratio is larger than the first watery feces ratio included in the reference database. Here, in a case where it is determined that the second watery feces ratio is equal to or less than the first watery feces ratio (NO in Step S21), the processing proceeds to Step S24.

On the other hand, in a case where it is determined that the second watery feces ratio is larger than the first watery feces ratio (YES in Step S21), in Step S22, the alert information generation part 324 generates second alert information indicating that the user is highly likely to have diarrhea.

Next, in Step S23, the alert information output part 325 outputs the second alert information generated by the alert information generation part 324 to the display device 5. The communication part 31 transmits the second alert information output by the alert information output part 325 to the display device 5. The display device 5 receives the second alert information and displays the received second alert information.

Next, in Step S24, the alert information generation part 324 calculates a second evacuation amount indicating an average evacuation amount per time in two weeks by using evacuation amount data acquired in two weeks.

Next, in Step S25, the alert information generation part 324 determines whether or not the second evacuation amount is smaller than the first evacuation amount included in the reference database. Here, in a case where it is determined that the second evacuation amount is equal to or larger than the first evacuation amount (NO in Step S25), the processing proceeds to Step S28.

On the other hand, in a case where it is determined that the second evacuation amount is smaller than the first evacuation amount (YES in Step S25), in Step S26, the alert information generation part 324 generates third alert information indicating that there is a high possibility that a meal intake amount of the user is insufficient.

Next, in Step S27, the alert information output part 325 outputs the third alert information generated by the alert information generation part 324 to the display device 5. The communication part 31 transmits the third alert information output by the alert information output part 325 to the display device 5. The display device 5 receives the third alert information and displays the received third alert information.

Next, in Step S28, the alert information generation part 324 calculates a second evacuation color ratio indicating a ratio of excretion of feces of a predetermined color in two weeks using evacuation color data acquired in two weeks. The predetermined color is, for example, black.

Next, in Step S29, the alert information generation part 324 determines whether or not the first evacuation color ratio and the second evacuation color ratio included in the reference database are different. Here, in a case where it is determined that the first evacuation color ratio and the second evacuation color ratio are the same (NO in Step S29), the processing proceeds to Step S32.

On the other hand, in a case where it is determined that the first evacuation color ratio and the second evacuation color ratio are different (YES in Step S29), in Step S30, the alert information generation part 324 generates fourth alert information indicating that a color of feces of the user changes.

Next, in Step S31, the alert information output part 325 outputs the fourth alert information generated by the alert information generation part 324 to the display device 5. The communication part 31 transmits the fourth alert information output by the alert information output part 325 to the display device 5. The display device 5 receives the fourth alert information and displays the received fourth alert information.

Next, in Step S32, the second data acquisition part 323 determines whether or not one week has elapsed from a time point at which it is determined that one week has elapsed last time. Here, in a case where it is determined that one week has not elapsed from a time point when it is determined that one week has elapsed last time (NO in Step S32), the processing proceeds to Step S44.

On the other hand, in a case where it is determined that one week has elapsed from a time point at which it is determined that one week has elapsed last time (YES in Step S32), in Step S33, the alert information generation part 324 calculates a second number of times of urination indicating an average number of times of urination per day in one week using excrement type data acquired in one week.

Next, in Step S34, the alert information generation part 324 determines whether or not the second number of times of urination is larger than the first number of times of urination included in the reference database. Here, in a case where it is determined that the second number of times of urination is equal to or less than the first number of times of urination (NO in Step S34), the processing proceeds to Step S37.

On the other hand, in a case where it is determined that the second number of times of urination is larger than the first number of times of urination (YES in Step S34), in Step S35, the alert information generation part 324 generates fifth alert information indicating that the user tends to have frequent urination.

Next, in Step S36, the alert information output part 325 outputs the fifth alert information generated by the alert information generation part 324 to the display device 5. The communication part 31 transmits the fifth alert information output by the alert information output part 325 to the display device 5. The display device 5 receives the fifth alert information and displays the received fifth alert information.

Next, in Step S37, the alert information generation part 324 determines whether or not the second number of times of urination is smaller than the first number of times of urination included in the reference database. Here, in a case where it is determined that the second number of times of urination is equal to or more than the first number of times of urination (NO in Step S37), the processing proceeds to Step S40.

On the other hand, in a case where it is determined that the second number of times of urination is smaller than the first number of times of urination (YES in Step S37), in Step S38, the alert information generation part 324 generates sixth alert information indicating that there is a high possibility that a water intake amount of the user is insufficient.

Next, in Step S39, the alert information output part 325 outputs the sixth alert information generated by the alert information generation part 324 to the display device 5. The communication part 31 transmits the sixth alert information output by the alert information output part 325 to the display device 5. The display device 5 receives the sixth alert information and displays the received sixth alert information.

Next, in Step S40, the alert information generation part 324 calculates a second number of times of bleeding indicating the number of times of bleeding in one week using bleeding data acquired in one week.

Next, in Step S41, the alert information generation part 324 determines whether or not the first number of times of bleeding included in the reference database is zero and the second number of times of bleeding is one or more. Here, in a case where it is determined that the first number of times of bleeding is not zero or the second number of times of bleeding is not one or more (NO in Step S41), the processing proceeds to Step S44.

On the other hand, in a case where it is determined that the first number of times of bleeding is zero and the second number of times of bleeding is one or more (YES in Step S41), in Step S42, the alert information generation part 324 generates eighth alert information indicating that there is a high possibility that the user is bleeding at the time of evacuation or urination.

Next, in Step S43, the alert information output part 325 outputs the eighth alert information generated by the alert information generation part 324 to the display device 5. The communication part 31 transmits the eighth alert information output by the alert information output part 325 to the display device 5. The display device 5 receives the eighth alert information and displays the received eighth alert information.

Next, in Step S44, the second data acquisition part 323 determines whether or not three days have elapsed from a time point at which it is determined that three days have elapsed last time. Here, in a case where it is determined that three days have not elapsed from a time point at which it is determined that three days have elapsed last time (NO in Step S44), the processing returns to Step S13.

On the other hand, in a case where it is determined that three days have elapsed from a time point at which it is determined that three days have elapsed last time (YES in Step S44), in Step S45, the alert information generation part 324 calculates a second specific gravity ratio indicating a ratio at which specific gravity of urine in three days is higher than a predetermined range using urine specific gravity data acquired in three days.

Next, in Step S46, the alert information generation part 324 determines whether or not the second specific gravity ratio is larger than the first specific gravity ratio included in the reference database. Here, in a case where it is determined that the second specific gravity ratio is equal to or less than the first specific gravity ratio (NO in Step S46), the processing returns to Step S13.

On the other hand, in a case where it is determined that the second specific gravity ratio is larger than the first specific gravity ratio (YES in Step S46), in Step S47, the alert information generation part 324 generates seventh alert information indicating that the user is highly likely to be dehydrated.

Next, in Step S48, the alert information output part 325 outputs the seventh alert information generated by the alert information generation part 324 to the display device 5. The communication part 31 transmits the seventh alert information output by the alert information output part 325 to the display device 5. The display device 5 receives the seventh alert information and displays the received seventh alert information.

As described above, first, in the first predetermined period, first data in which excretion related data of the user acquired by the excretion sensor 1 installed in a toilet is associated with a user ID for identifying the user is acquired. Then, a reference database indicating an excretion tendency of the user is generated on the basis of the first data acquired in the first predetermined period. After the above, in the second predetermined period, second data in which excretion related data of the user acquired by the excretion sensor 1 installed in a toilet is associated with a user ID is acquired. Then, alert information to the user is generated on the basis of the reference database and the second data acquired in the second predetermined period, and the generated alert information is output.

Therefore, general statistical data and a current excretion tendency of the user in the second predetermined period are not compared, and a past excretion tendency of the user in the first predetermined period and a current excretion tendency of the user in the second predetermined period are compared. Accordingly, it is possible to detect a change in an excretion tendency of the user himself or herself and output alert information according to a change in a health condition of an individual user.

Note that, in the present embodiment, the alert information output part 325 outputs each of the first to eighth alert information every time each of the first to eighth alert information is generated, but the present disclosure is not particularly limited to this, and the generated first to eighth alert information may be collectively output.

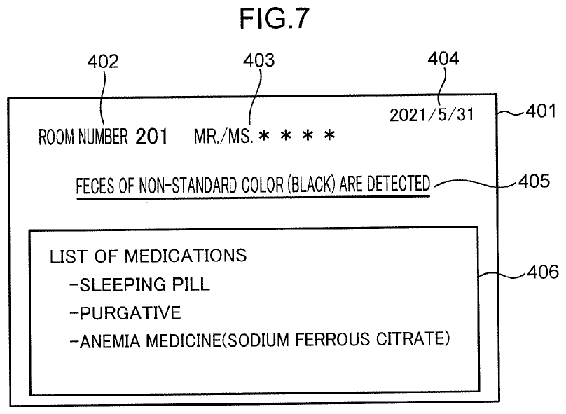

FIG. 7 is a diagram illustrating an example of a display screen 401 of fourth alert information displayed on the display device 5.

The display screen 401 includes display fields 402 to 406. The display field 402 displays a room number of the corresponding user. Note that since the user lives in a facility, a room number is displayed in the display field 402. The display field 403 displays a name of the corresponding user. The display field 404 displays date and time when alert information is output. The display field 405 displays fourth alert information indicating that feces of a non-standard color (black) are detected. The display field 406 displays related data related to fourth alert information. Here, a type of medicine taken by the user is displayed as related data.

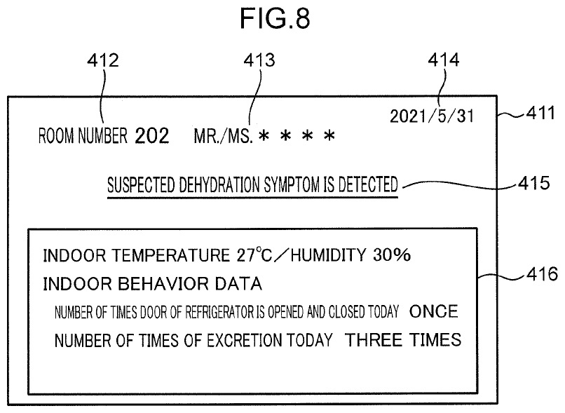

FIG. 8 is a diagram illustrating an example of a display screen 411 of seventh alert information displayed on the display device 5.

The display screen 411 includes display fields 412 to 416. The display field 412 displays a room number of the corresponding user. Note that since the user lives in a facility, a room number is displayed in the display field 412. The display field 413 displays a name of the corresponding user. The display field 414 displays date and time when alert information is output. The display field 415 displays seventh alert information indicating that a suspected dehydration symptom is detected. The display field 416 displays related data related to seventh alert information. Here, temperature and humidity of a room where the user mainly lives, the number of times of opening and closing of a refrigerator today, and the number of times of excretion of the user today are displayed as related data.

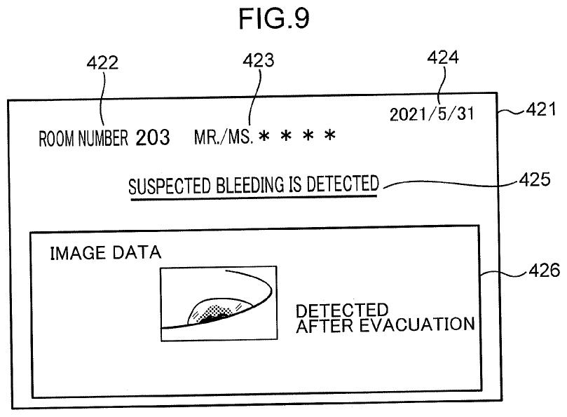

FIG. 9 is a diagram illustrating an example of a display screen 421 of eighth alert information displayed on the display device 5.

The display screen 421 includes display fields 422 to 426. The display field 422 displays a room number of the corresponding user. Note that since the user lives in a facility, a room number is displayed in the display field 422. The display field 423 displays a name of the corresponding user. The display field 424 displays date and time when alert information is output. The display field 425 displays eighth alert information indicating that suspected bleeding is detected. The display field 426 displays related data related to eighth alert information. Here, image data acquired when bleeding is detected and a bleeding start timing are displayed as related data. The bleeding start timing indicates whether bleeding is detected before evacuation, during evacuation, after evacuation, before urination, during urination, or after urination.

Note that the reference database generation part 322 may periodically update a reference database after generating the reference database using excretion related data for one month. For example, the reference database generation part 322 may update a reference database using excretion related data for one month every month. Further, for example, after generating a reference database using excretion related data for one month, the reference database generation part 322 may update the reference database using excretion related data for three months every three months longer than one month.

In each of the above embodiments, each constituent element may be implemented by being configured with dedicated hardware or by execution of a software program suitable for each constituent element. Each constituent element may be implemented by a program execution part, such as a CPU or a processor, reading and executing a software program recorded in a recording medium such as a hard disk or a semiconductor memory. Further, the program may be carried out by another independent computer system by being recorded in a recording medium and transferred or by being transferred via a network.

Some or all functions of the device according to the embodiment of the present disclosure are implemented as large scale integration (LSI), which is typically an integrated circuit. These may be individually integrated into one chip, or may be integrated into one chip so as to include some or all of them. Further, circuit integration is not limited to LSI, and may be implemented by a dedicated circuit or a general-purpose processor. A field programmable gate array (FPGA), which can be programmed after manufacturing of LSI, or a reconfigurable processor in which connection and setting of circuit cells inside LSI can be reconfigured may be used.

Further, some or all functions of the device according to the embodiment of the present disclosure may be implemented by a processor such as a CPU executing a program.

Further, all numbers used above are illustrated to specifically describe the present disclosure, and the present disclosure is not limited to the illustrated numbers.

Further, order in which steps illustrated in the above flowchart are executed is for specifically explaining the present disclosure, and may be any order other than the above order as long as a similar effect is obtained. Further, some of the above steps may be executed simultaneously (in parallel) with other steps.

### INDUSTRIAL APPLICABILITY

Since the technique according to the present disclosure can output alert information according to a change in a health condition of an individual user, the technique according to the present disclosure is useful as a technique for outputting alert information to the user.

1. An information processing method comprising:
by a computer,
acquiring first data in which excretion related data of a user acquired by a sensor installed in a toilet and a user ID for identifying the user are associated with each other in a first predetermined period,
generating a reference database indicating an excretion tendency of the user based on the acquired first data;
acquiring second data in which the excretion related data of the user acquired by the sensor installed in the toilet and the user ID are associated with each other in a second predetermined period;
generating alert information to the user based on the reference database and the second data; and
outputting the generated alert information.

2. The information processing method according to claim 1, wherein
the excretion related data includes feces shape data indicating whether a shape of excreted feces is hard feces, normal feces, or watery feces,
the reference database includes a first hard feces ratio indicating a ratio of the hard feces excreted among all evacuations in the first predetermined period, and
in generation of the alert information, a second hard feces ratio indicating a ratio of the hard feces excreted among all evacuations in the second predetermined period is calculated, and in a case where the second hard feces ratio is higher than the first hard feces ratio, first alert information indicating that the user is highly likely to have constipation is generated.

3. The information processing method according to claim 1, wherein
the excretion related data includes feces shape data indicating whether a shape of excreted feces is hard feces, normal feces, or watery feces,
the reference database includes a first watery feces ratio indicating a ratio of the watery feces excreted among all evacuations in the first predetermined period, and
in generation of the alert information, a second watery feces ratio indicating a ratio of the watery feces excreted among all evacuations in the second predetermined period is calculated, and in a case where the second watery feces ratio is higher than the first watery feces ratio, second alert information indicating that the user is highly likely to have diarrhea is generated.

4. The information processing method according to claim 1, wherein
the excretion related data includes evacuation amount data indicating an evacuation amount,
the reference database includes a first evacuation amount indicating an average evacuation amount per time in the first predetermined period, and
in generation of the alert information, a second evacuation amount indicating an average evacuation amount per time in the second predetermined period is calculated, and in a case where the second evacuation amount is smaller than the first evacuation amount, third alert information indicating that there is a high possibility that a meal intake amount of the user is insufficient is generated.

5. The information processing method according to claim 1, wherein
the excretion related data includes evacuation color data indicating a color of excreted feces,
the reference database includes a first evacuation color ratio indicating a ratio of excretion of feces of a predetermined color among all evacuations in the first predetermined period, and
in generation of the alert information, a second evacuation color ratio indicating a ratio of excretion of feces of the predetermined color among all evacuations in the second predetermined period is calculated, and in a case where the first evacuation color ratio and the second evacuation color ratio are different, fourth alert information indicating that a color of feces of the user changes is generated.

6. The information processing method according to claim 1, wherein
the excretion related data includes excrement type data indicating that excrement of the user is urine,
the reference database includes a first number of times of urination indicating an average number of times of urination per day in the first predetermined period, and
in generation of the alert information, a second number of times of urination indicating an average number of times of urination per day in the second predetermined period is calculated, and in a case where the second number of times of urination is larger than the first number of times of urination, fifth alert information indicating that the user tends to have frequent urination is generated.

7. The information processing method according to claim 1, wherein
the excretion related data includes excrement type data indicating that excrement of the user is urine,
the reference database includes a first number of times of urination indicating an average number of times of urination per day in the first predetermined period, and
in generation of the alert information, a second number of times of urination indicating an average number of times of urination per day in the second predetermined period is calculated, and in a case where the second number of times of urination is smaller than the first number of times of urination, sixth alert information indicating that there is a high possibility that a water intake amount of the user is insufficient is generated.

8. The information processing method according to claim 1, wherein
the excretion related data includes urine specific gravity data indicating whether or not specific gravity of urine of the user is higher than a predetermined range,
the reference database includes a first specific gravity ratio indicating a ratio in which specific gravity of the urine is higher than the predetermined range among all urinations in the first predetermined period, and
in generation of the alert information, a second specific gravity ratio indicating a ratio in which specific gravity of the urine is higher than the predetermined range among all urinations in the second predetermined period is calculated, and in a case where the second specific gravity ratio is higher than the first specific gravity ratio, seventh alert information indicating that the user is highly likely to be dehydrated is generated.

9. The information processing method according to claim 1, wherein
the excretion related data includes bleeding data indicating that the user bleeds at a time of evacuation or urination,
the reference database includes a first number of times of bleeding indicating the number of times of bleeding in the first predetermined period, and
in generation of the alert information, a second number of times of bleeding indicating the number of times of bleeding in the second predetermined period is calculated, and in a case where the first number of times of bleeding is smaller than a predetermined number of times and the second number of times of bleeding is equal to or more than a predetermined number of times, eighth alert information indicating that there is a high possibility that the user is bleeding at the time of evacuation or urination is generated.

10. The information processing method according to claim 1, wherein
the excretion related data includes evacuation related data related to evacuation of the user and urination related data related to urination of the user, and
the second predetermined period for acquiring the evacuation related data is longer than the second predetermined period for acquiring the urination related data.

11. The information processing method according to claim 1, wherein
in generation of the alert information, related data related to the generated alert information is further acquired, and
in output of the alert information, the related data is output together with the alert information.

12. The information processing method according to claim 11, wherein
the related data includes a type of medicine taken by the user.

13. The information processing method according to claim 11, wherein
the related data includes at least one of meal content of the user, an environment in a house of the user, and an activity amount of the user.

14. An information processing device comprising:
a first data acquisition part that acquires first data in which excretion related data of a user acquired by a sensor installed in a toilet and a user ID for identifying the user are associated with each other in a first predetermined period;
a reference database generation part that generates a reference database indicating an excretion tendency of the user based on the acquired first data;
a second data acquisition part that acquires second data in which the excretion related data of the user acquired by the sensor installed in the toilet and the user ID are associated with each other in a second predetermined period;
an alert information generation part that generates alert information to the user based on the reference database and the second data; and
an output part that outputs the generated alert information.

15. A non-transitory computer readable recording medium storing an information processing program that causes a computer to function to
acquire first data in which excretion related data of a user acquired by a sensor installed in a toilet and a user ID for identifying the user are associated with each other in a first predetermined period,
generate a reference database indicating an excretion tendency of the user based on the acquired first data,
acquire second data in which the excretion related data of the user acquired by the sensor installed in the toilet and the user ID are associated with each other in a second predetermined period,
generate alert information to the user based on the reference database and the second data, and
output the generated alert information.
